(19) United States
(12) Patent Application Publication
(10) Pub. No.: US 2014/0011894 A1
(43) Pub. Date: Jan. 9, 2014

(54) Stable Emulsions of Personal Care Products

(71) Applicant: Kemin Industries, Inc., Des Moines, IA (US)

(72) Inventor: Vandana Srivastava, Johnston, IA (US)

(73) Assignee: Kemin Industries, Inc., Des Moines, IA (US)

(21) Appl. No.: 13/895,716

(22) Filed: May 16, 2013

Related U.S. Application Data
(60) Provisional application No. 61/648,130, filed on May 17, 2012.

(51) Int. Cl. A61K 8/97; A61Q 19/00; A61K 8/55

(52) U.S. Cl. CPC A61K 8/97; A61K 8/553; A61Q 19/007
USPC 514/783

(57) ABSTRACT

Natural extracts of spearmint and rosemary, together with hydrolyzed lecithin, are used to formulate a natural tinted moisturizer with more that 96% natural/nature-derived raw materials. Color pigments are suspended in the emulsion system thickened by xanthan gum. The process uses a combination of mixing with a homogenizer and a multi-speed stirrer. Stability is observed in accelerated conditions. The product results with an excellent emulsion stability. The hydrolyzed lecithin is liquid at room temperature. This study demonstrates that it is an easy to use emulsifier in cold-processed emulsions with a thickener system that can be dispersed and activated at room temperature. The hydrolyzed lecithin was used to emulsify blends of oils into a stable lotion for the cosmetic and personal care markets. The emulsion was found to be highly stable when centrifuged and studied at accelerated stability conditions

This application claims priority to U.S. Patent Application Ser. No. 61/648,130, filed May 17, 2012.

### BACKGROUND OF THE INVENTION

The present invention relates generally to emulsions and, more specifically, to stable emulsions particularly suited to the personal care industry.

Most personal care lotions are oil in water (o/w) emulsions made in unit batch processes, requiring heat to melt waxes, pellets, emulsifiers and other ingredients that are solid at room temperature. A commonly used 500 gallon stainless steel jacketed vessel used in production of batch processes can typically take 1-2 hours to heat to emulsification temperature and an equal amount to cool down to room temperature to allow handling and packaging. A large amount of energy in the form of steam and cold-water is required in these processes. Steam handling also raises personnel safety concerns. A process that eliminates the heating and cooling cycle can impart a benefit in reducing costs with respect to energy and time, reduce safety concerns and allow introduction of heat-sensitive raw-materials in the finished product.

Color cosmetics are integral part of the personal care regime. Following the current trend in the market, color cosmetics have started to see an increase in demand for natural or nature-derived products. The mineral make-up market is a classic example of this boom. Raw materials offered by Kemin Personal Care (KPC) have the advantage of being derived completely from natural and non-genetically modified organism (non-GMO) sources. Lysofix™, a natural emulsifier, Speramox™ a natural water-dispersible antioxidant, and Rosamox™, a natural oil-soluble antioxidant, are desirable raw materials to be used in a natural formulation. The addition of these raw materials to any formulation increases the percentage of natural materials used in a product seeking to make ‘natural’ claims. The functional benefit of these raw materials is extended to various color cosmetic products such as foundations, powders, mascara, liners, tinted moisturizer and the like. These products can be emulsions, suspensions, and cakes or blends of powders with natural or organic color pigments or the like.

### SUMMARY OF THE INVENTION

The present invention consists of stable emulsions containing a high proportion of natural ingredients particularly suited for the personal care industry. The emulsions include an emulsifier containing hydrolyzed lecithin that is liquid at room temperature. The liquid emulsifier allows the formation of emulsions without the use of elevated temperatures, i.e., cold processed emulsions. Such emulsions are easier, safer and less expensive to prepare and are less likely to cause damage to heat labile components of the emulsion. The emulsifier also has been found to create an emulsion that is stable over long periods of time and at high temperatures, resulting in an emulsion that is suitable for personal care products containing pigments.

### DETAILED DESCRIPTION OF PREFERRED EMBODIMENTS

Emulsions may be oil-in-water emulsions or water-in-oil emulsions. Since many personal care products contain both a water phase and an oil phase, many personal care products are emulsions. Examples of such products include creams, moisturizers, foundations, mascara, liners and the like.

Personal care products need to be stable over extended shelf life periods and over expected temperature cycles such that there is little or no detectable separation of the components of the emulsified products. Further, such products must be protected against degradation and spoilage over the expected shelf life, typically by the addition of antioxidants and antimicrobials. In addition, the high demand for “natural” ingredients places a priority on maximizing such components that can be labeled “natural.” Sources of natural antioxidants and antimicrobials include the natural extracts of plants, including specifically plants of the family Lamiaceae, including but not limited to mint, rosemary, sage, savory, marjoram, oregano, hyssop, thyme, lavender, and perilla.

One emulsifier suitable for use in the stable emulsions of the present invention includes hydrolyzed lecithin or lysophosphatidylcholines, wherein the lecithin has been extracted without the use of chemical solvents from non-genetically modified soybeans, eggs, milk, rapeseed, cottonseed, and sunflower (non-GMOs). Preferably, the lysophosphatidylcholines are mixed with a suitable natural carrier in an amount between about 1% and about 20% by weight and preferably between about 2% and about 5%. An example of a suitable carrier is glycerin. Preferably, the emulsifier is a flowable liquid at room temperature.

### EXAMPLE 1

A water/oil (w/o) emulsion system was chosen to suspend the color pigments. Lysofix (Kemin Industries, Inc., Des Moines, Iowa) was used as emulsifier. Lysofix™ is a natural emulsifier for use in personal care products. It is a 3% solution of lysophosphatidylcholines (LPC) derived from soybean dissolved in glycerin derived from a vegetable source. It is a clear liquid, amber in color with a viscosity between 3,500 cP and 4,500 cP at 20° C. It flows slowly but easily by itself, and becomes more free-flowing when mixed with water. Speramox (Kemin) and Rosamox (Kemin) were added to the system to increase oxidative stability of the product in the presence of organic pigments. Xanthan gum (UltraPure Chemicals Inc.) was used as a thickener, Crodafos CS20A (Croda Inc., Edison, N.J.) was used as a thickening and stabilizing emulsifier. Olive oil (Sigma Aldrich, St. Louis, Mo.) and safflower oil (Kemin Health, Des Moines, Iowa) were used as natural emollients. Crodamol STS (Croda Inc.) and Cetiol C5 (BASF, New Milford, Conn.) were used as dry-feel emollients to reduce tackiness. Apart from choosing natural/nature-derived materials, the challenge was to suspend color-imparting pigments in an emulsion structure to prevent settling through the life cycle of the product. In addition, a product that rubs in into a smooth, uniform and non-tacky film on the skin was preferred. On skin, the pigments were expected to feather evenly, not to leave a demarcation line, and not to transfer to clothes. The product had to be aesthetically attractive and stable under normal stress conditions.

Four shades of color pigment blends from Maybelline, NY (PD11009.01-04), and one shade from Cover Girl, UK (PD 11009.06), were used to make similar formulation. Light shade from Maybelline, NY and Dark shade from Cover Girl, UK demonstrated stability characteristics similar to the current formulation.

### Materials and Methods

Raw Materials. Table 1 lists the materials used in the preparation of natural tinted moisturizer. A Color Pigment Blend (Mineral Power Natural Perfecting Powder Foundation, Classic Ivory Light 2, Maybelline, NY) was purchased and used as standard color pigment source. The components of this blend are listed in Table 2. Apart from these materials, deionized water from Millipore was obtained in-house.

TABLE 1
Samples and chemicals used in preparation of Natural Tinted Moisturizer

| | Brand/Common | | |
|---|---|---|---|
| Ingredient [INCI] | Name | Grade/Source/Certification | Supplier/Manufacturer |
| Xanthan Gum | Xanthan Gum | NF/Natural | Ultrapure Chemical Inc., |
| | | | Red Bank, NJ |
| Sodium Hydroxide | Sodium | NF/Synthetic | Spectrum Chemicals & |
| | Hydroxide | | Laboratory Products, |
| | Pellets | | New Brunswick, NJ |
| Methylparaben | Methylparaben | Food Grade/Synthetic | Kemin Food |
| | | | Technologies, Des |
| | | | Moines, IA |
| Glycerin (and) Glycine | Lysofix ™ | Personal Care/Natural | Kemin Personal Care, |
| Max (Soybean) Seed | | | Des Moines, IA |
| Extract | | | |
| Maltodextrin (and) | Speramox ™ | Personal Care/Natural | Kemin Personal Care, |
| Mentha Spicata Leaf | | | |
| Extract | | | |
| Olea Europaea (Olive) | Refined Olive | Analytical Grade/Natural | Sigma-Aldrich, St. |
| Fruit Oil | Oil | | Louis, MO |
| Carthamus Tinctorius | Safflower Oil | Food Grade/Natural | Kemin Health, Des |
| (Safflower) Seed Oil | | | Moines, IA |
| Stearyl Alcohol | Stearyl Alcohol | NF | Spectrum Chemicals |
| Cetearyl Alcohol (and) | Crodafos ™ | Personal | Croda Inc., Edison NJ |
| Ceteth-20 Phosphate | CS20A | Care/Natural/Ecocert | |
| (and) Dicetyl | | | |
| Phosphate | | | |
| PPG-3 Benzyl Ether | Crodamol ™ | Personal Care/ | Croda Inc. |
| Myristate | STS | Natural/Ecocert | |
| Coco Caprylate | Cetiol ™ C5 | Personal Care/ | GMZ Inc., West |
| | | Natural/Ecocert | Chester, OH/BASF, |
| | | | New Milford, CT |
| Propylparaben | Propylparaben | Food Grade/Synthetic | Kemin Food |
| | | | Technologies |
| Helianthus Annuus | Rosamox ™ | Personal Care/Natural | Kemin Personal Care, |
| Seed Extract (and) | | | |
| Rosmarinus Officinalis | | | |
| Leaf Extract | | | |
| Fragrance | Chemia #47212 | Personal Care/Synthetic | Chemia Corporation, St. |
| | | | Louis, MO |
| Color Pigment Blend* | Covergirl | Personal Care | Walgreens, Des Moines, |
| | trublend | | IA/Proctor & Gamble, |
| | minerals 405; | | Surrey, UK |
| | translucent fair | | |

*INCI name listed in Table 2

TABLE 2
INCI names of components of Color Pigment Blend*

| Drug Facts | Ingredient [INCI] |
|---|---|
| Ingredient | Talc |
| | Mica |
| May Contain | Avena Sativa (Oat) Kernel |
| | Flour |
| | Zinc Stearate |
| | Calcium Silicate |
| | Titanium Dioxide |
| | Mineral Oil |
| | Glycerin |
| | Quaternium-15 |
| | Methylparaben |
| | Methicone |
| | Propylparaben |
| | Sodium dehydroacetate |
| | BHT |
| | Isopropyl Myristate |
| | Iron Oxides |
| | Carmine |
| | Yellow 5 Lake |
| | Ultramarines |

Equipment. Table 3 lists the equipment that was used in the preparation and testing of the natural tinted moisturizer.

TABLE 3
Equipment used for preparation and testing of emulsion.

| Item | Manufacturer/Model |
|---|---|
| Multi-speed stirrer | IKA/RW20 Digital |
| Multi-speed | Carter Motor |
| homogenizer | Company/MUD1001AN |
| Water Bath | Fisher Scientific/Isotemp 210 |
| | Digital Control |
| Hotplate | Fischer Scientific/Isotemp Digital |
| Viscometer | Brookfield/DVII+ |
| pH meter | Orion/520A |
| Centrifuge | Eppendorf/Centrifuge 5810 |
| Microscope | Motic/BA400 |
| Incubator | Infors/Multitron |
| Refrigerator Freezer | Whirlpool/ET1WTK |

Formulation. Table 4 lists the percentages of raw materials and phases of formulations used to make Natural Tinted Moisturizer.

TABLE 4
Formulation with phases, percentage, and amount used [PD11009.05
Light].

| | | | PD11009.05 |
|---|---|---|---|
| | | | Light |
| | | PD11009.05 | Weight (g) |
| | | Light | used for |
| Phase | Ingredient | % w/w | 250 g |
| A | Deionized Water | 59.15 | 135.38 |
| | Xanthan Gum | 0.50 | 1.25 |
| | Sodium Hydroxide | 0.20 | 0.50 |
| | Methylparaben | 0.12 | 0.30 |
| B | Deionized Water | — | 12.50 |
| | Glycerin (and) Glycine Max | 5.00 | 12.50 |
| | (Soybean) Seed Extract | | |
| | Maltodextrin (and) Mentha Spicata | 0.25 | 0.63 |
| | Leaf Extract | | |
| C | Olea Europaea (Olive) Fruit Oil | 5.00 | 12.50 |
| | Carthamus Tinctorius (Safflower) | 5.00 | 12.50 |
| | Seed Oil | | |
| | Stearyl Alcohol | 3.00 | 7.50 |
| | Cetearyl Alcohol (and) Ceteth-20 | 7.00 | 17.50 |
| | Phosphate (and) Dicetyl Phosphate | | |
| | PPG-3 Benzyl Ether Myristate | 5.00 | 12.50 |
| | Color Pigment Blend* | 5.00 | 12.50 |
| | Coco Caprylate | 4.00 | 10.00 |
| | Propylparaben | 0.03 | 0.08 |
| D | Helianthus Annuus (Sunflower) | 0.25 | 0.63 |
| | Seed Extract (and) Rosmarinus | | |
| | Officinalis (Rosemary) Leaf Extract | | |
| E | Fragrance | 0.50 | 1.25 |
| F | Citric Acid (33.33% solution) | q.s. to pH | 3.10 |
| | | 5 ± 0.5 | |
| G | Deionized Water (includes water | q.s. to 100% | 26.73 |
| | lost in evaporation) | | |
| Total | | 100.00 | 250.00 |
| | Total % of natural/nature-derived | 96.65 | |
| | raw-materials | | |

*INCI name listed in Table 2.

Product Preparation. Deionized Water of Phase A was weighed in a 500 mL side glass beaker. The multi-speed stirrer with a 1.5 inch marine propeller head was positioned in the water and the speed adjusted to form a vortex (800 RPM). Weighed amount of xanthan gum was sprinkled in the vortexing liquid in a way that no clumps were formed. Sodium hydroxide was added to make the environment alkaline. This mixture was stirred for 30 minutes to facilitate complete hydration of the gum. Ingredients of Phase B were weighed in a 100 mL side glass beaker. Ingredients of Phase C were weighed in a 600 mL main glass beaker with a known tare weight. After the ingredients of Phase A were completely hydrated (30 minutes), Phase A, B and C were heated to 82° C. in pre-heated water bath. Main beaker with heated Phase C was positioned under the homogenizer and heated Phase A and B were added consequently. The homogenizer shaft was lowered and positioned to be completely covered by liquid. The batch was homogenized at regulator speed of 45 for 10 minutes. The shaft was removed and the emulsion scraped cleaned from the blades. The main beaker was positioned under the multi-speed stirrer and the emulsion was allowed to cool while being mixed (1500 RPM). At 60° C., Phase D was added to the main beaker and allowed to mix. At 45° C., Phase E was added and allowed to mix. The main beaker was removed from the stirrer and the pH measured. The pH was found to be 7.50 at 45° C. To adjust the pH to 5±0.5, Phase F was added in small amounts, the emulsion mixed with a glass rod and the pH measured. The resultant pH was found to be 5.11. The main beaker was then weighed and Phase G added to balance the total weight of product to 250 g. The main beaker was positioned again under the multi-speed stirrer and the emulsion was allowed to be mixed until smooth, uniform and cool (30° C.).

Product Analysis. The appearance, color, odor of sample was analyzed visually. The application characteristic was analyzed by applying a dab on the volar fore-arm, rubbing the product in with index-finger and checking the tackiness after product was completely absorbed on skin. Viscosity was measured on the sample kept undisturbed at 20° C. overnight in a glass beaker (Corning, 150 mL). Spindle S 04 was used at 20 RPM on Brookfield Viscometer. Manual readings of viscosity and torque were taken after one, two, and three minutes. The same sample was used to measure pH using the pH meter. A small amount of sample was taken on a microscope glass slide (Fisher Scientific) and carefully covered by a glass cover-slip (Fisher Scientific). This slide was observed under the microscope with 40× magnification.

Centrifuge Stability. Sample was transferred into two disposable conical tubes (Corning, 15 mL) to a total weight of 17 g. One tube was heated in a water-bath to 40° C. Both tubes were then centrifuged at 4000 RPM for 30 minutes.

Accelerated Stability. Sample was transferred in two disposable conical tubes (Corning, 50 mL). One tube was marked and kept in the incubator set at 45° C. Visual and qualitative readings on appearance, color, odor and stability of emulsion were taken on this sample at one week, two weeks, three weeks, one month, two months and three months data points. The other tube was kept in the freezer at −12° C. for 20 hours, and returned to the lab counter at room temperature (RT) for 4 hours. Visual and qualitative readings were taken on appearance, color, odor and stability of emulsion. The sample was then kept in the incubator at 45° C. for 20 hours and returned to the lab counter for 4 hours. Visual and qualitative readings were taken again on appearance, color, odor and stability of emulsion. This cycle was repeated for five times.

### Results

Initial Physical Characteristics. The appearance of the Natural Tinted Moisturizer after preparation was smooth, uniform, and tan in color. The application on skin was easy with no residual tackiness. The moisturizer imparted a uniform color deposition on skin. The fragrance masked any raw-material odor and had a pleasant chocolate-like top note. The resultant pH was 5.11. The moisturizer had a viscosity of 10,240 cP at 20° C. The samples centrifuged at RT and 40° C. did not show any phase separation or creaming (Table 5). There was a minor settling of pigments at the bottom. The sample observed under the microscope showed a uniform structure with pigments of various shapes and sizes interspersed in the body of the emulsion.

TABLE 5
Physical Characteristics of Natural Tinted Moisturizer (Light)

| Attribute | Observation |
|---|---|
| Appearance | Smooth and uniform |
| Color (Visual) | Tan |
| Odor | Pleasant |
| Application | Easy spreading, leaves a uniform |
| | residue on skin |
| pH | 5.11 at 20° C. |
| Centrifuge | No separation of phases, minor |
| (RT) | settling, no creaming or |
| | coalescence |
| Centrifuge | No separation of phases, minor |
| (40° C.) | settling, no creaming or |
| | coalescence |
| Viscosity | 10,240 cP at 20° C.; 3 minutes, 20 RPM, |
| | 51.2% torque using |
| | spindle S04, |

Accelerated Stability Data. The sample did not demonstrate any phase separation, creaming or settling of pigments over the three months at 45° C. It did not show any change in appearance or color when observed visually. The sample showed excellent physical stability when centrifuged after incubation at 45° C. for a month. The samples undergoing F/T cycles showed no change in appearance, and color when observed visually. There was no phase separation, creaming or settling of pigments observed in the sample undergoing F/T cycle (Table 6).

TABLE 6
Accelerated Stability Data

| | Period of | |
|---|---|---|
| Condition | observations | Observation |
| Incubation at 45° C. | One, two, three | No change was observed in |
| | weeks, one, two | appearance, color, and |
| | and three | odor. No phase separation, |
| | months | settling or creaming was |
| | | observed. There was no |
| | | visual change in the flow |
| | | ability of the product. |
| Freeze/Thaw (F/T) | Five cycles | No change was observed in |
| cycle | | appearance and color. After |
| | | two F/T cycles, there was |
| | | change in the odor; the note |
| | | gradually changed from the |
| | | chocolate-like to calamine |
| | | pigment (metallic). By the |
| | | end of five F/T cycles, the |
| | | metallic odor had become |
| | | more prominent. However, |
| | | there was no phase |
| | | separation, settling or |
| | | creaming observed in the |
| | | emulsion. |

### Discussion

This exercise demonstrates that more than 96% of natural/nature-derived raw-materials can be used to make a stable and attractive tinted moisturizer. Lysofix proved to be an effective emulsifier; contributing to the structural integrity of the emulsion when used in conjunction with a co-emulsifier and thickening system. The sample showed no change in the emulsion characteristic. This indicates that the formulation was effective in suspending the color-pigments in the emulsion, and no settling of pigments is expected over the product life-cycle for over 24 months at RT.

### EXAMPLE 2

Emulsions are thermodynamically unstable mixtures of immiscible materials like oil/waxes and water. Emulsifiers delay separation of the immiscible materials by forming interfacial films, resulting in oil in water (o/w) or water in oil (w/o) emulsions. Cosmetic emulsions range from low-viscosity milks to high-viscosity creams. Lysofix offers a natural alternative to scores of synthetic emulsifiers available in a market where consumers are inclined toward choosing products made with natural materials.

The objective was to study the behavior of Lysofix as a single emulsifier in a gel-based emulsion made at room temperature. Previous processes using either heat or a homogenizer had been studied to compare the characteristics of the resulting emulsion. Emulsions were found to be formed in all cases. The emulsion with heat was found to be darker in color and required more time to prepare. The emulsion made with a homogenizer was found to be lighter in color but had reduced viscosity, even though the time required to prepare the emulsion was far less than the process with and without heat. Previous work included identification of a thickener system that worked at room temperature (RT). Xanthan gum (Sigma-Aldrich, St. Louis, Mo.) and Magnesium Aluminum Silicate (Veegum Pure, R.T. Vanderbilt Company Inc., Norwalk, Conn.) did not stabilize the emulsion made at RT. Carbomer (Lubrizol Advanced Materials, Cleveland, Ohio) did not stabilize the emulsion when used less than 0.5%. Studies were done earlier to identify a blend of most compatible polar and non-polar oils. Sunflower oil, jojoba oil and dimethicone were used but eliminated from the study due to instability, color and compatibility issues.

### Materials and Methods

Raw Materials. Table 7 lists the materials used in the preparation of emulsion. Apart from the materials used in Table 7, deionized water from Millipore was obtained in-house.

TABLE 7
Samples and chemicals used in preparation of emulsion

| | Brand/Common | | |
|---|---|---|---|
| Ingredient [INCI] | Name | Grade | Supplier/Manufacturer |
| Carbomer | Carbomer 940 | Personal | Lubrizol Advanced |
| | | Care | Materials, Cleveland, |
| | | | OH |
| Carthamus Tinctorius | Safflower Oil | Food Grade | Kemin Health, Des |
| (Safflower) Seed oil | | | Moines, IA |
| Olea Europaea (Olive) | Refined Olive | Analytical | Sigma-Aldrich, St. Louis, |
| Fruit Oil | Oil | Grade | MO |
| Coco-Caprylate | Cetiol C5 | Personal | GMZ Inc, West Chester, |
| | | Care | OH/BASF, New Milford, |
| | | | CT |
| Phenoxyethanol (and) | Optiphen ™ | Personal | ISP Technologies, Inc, |
| Caprylyl Glycol | | Care | Columbus, OH |
| Glycerin (and) Glycine | Lysofix ™ | Personal | Kemin Personal Care, |
| Max (Soybean) Seed | | Care | Des Moines, IA |
| Extract | | | |
| Sodium Hydroxide | Sodium | NF | Spectrum Chemicals & |
| | Hydroxide | | Laboratory Products, |
| | Pellets | | New Brunswick, NJ |

Equipment. Table 8 lists the equipment that was used in the preparation and testing of emulsions formed.

TABLE 8
Equipment used for preparation and testing of emulsion.

| Item | Manufacturer/Model |
|---|---|
| Multi-speed stirrer | IKA/RW20 Digital |
| Viscometer | Brookfield/DVII+ |
| pH meter | Orion/520A |
| Colorimeter | Hunter Lab/Colorflex 45/0 |
| Centrifuge | Eppendorf/Centrifuge 5810 |
| Incubator | Infors/Multitron |
| Refrigerator Freezer | Whirlpool/ET1WTK |

Formulation. Table 9 lists the percentages of raw materials and phases of formulation used to make cold processed emulsion.

TABLE 9
Emulsion formulation with phases and percentage of raw materials
[PD11003.18].

| | | | | | Control |
|---|---|---|---|---|---|
| | | | PD11003.18 | | Weight |
| | | PD11003.18 | Weight used | Control | used for |
| Phase | Ingredient | % w/w | for 250 gm | % w/w | 250 gm |
| A | Deionized | 71.75 | 166.87 | 78.75 | 180.87 |
| | Water | | | | |
| | Carbomer | 0.50 | 1.25 | 0.50 | 1.25 |
| B | Glycerin | 7.00 | 17.50 | 0.00 | 0.00 |
| | (and) | | | | |
| | Glycine Max | | | | |
| | (Soybean) | | | | |
| | Seed | | | | |
| | Extract | | | | |
| | Deionized | — | 12.50 | — | 12.50 |
| | Water | | | | |
| C | Carthamus | 12.00 | 30.00 | 12.00 | 30.00 |
| | Tinctorius | | | | |
| | (Safflower) | | | | |
| | Seed | | | | |
| | oil | | | | |
| | Olea | 6.00 | 15.00 | 6.00 | 15.00 |
| | Europaea | | | | |
| | (Olive) | | | | |
| | Fruit Oil | | | | |
| D | Coco- | 2.00 | 5.00 | 2.00 | 5.00 |
| | Caprylate | | | | |
| | Phenoxy- | 0.60 | 1.50 | 0.60 | 1.50 |
| | ethanol | | | | |
| | (and) | | | | |
| | Caprylyl | | | | |
| | Glycol | | | | |
| E | Sodium | 0.15 | 0.38 | 0.15 | 0.38 |
| | Hydroxide | | | | |
| | (33.33%) | | | | |
| F | Deionized | q.s. to 100% | 10.00 | q.s. to 100% | 14.00 |
| | Water | | | | |
| | (includes | | | | |
| | water | | | | |
| | lost in | | | | |
| | transfer) | | | | |

Emulsion Preparation. Deionized water of Phase A (Table 9) was weighed in a 500 mL glass beaker (referred to later as the main beaker) with known tare weight. The multi-speed stirrer with a 1.5 inch marine propeller head was adjusted in the liquid and the speed adjusted to form a vortex (800 RPM). A weighed amount of Carbomer was sprinkled into the vortexing liquid to avoid agglomeration. This mixture was stirred for 30 minutes to form a uniform dispersion. Weighed ingredients of Phase B, and Phase C (Table 9) were added to the main beaker. The speed was increased to adjust to the increase in viscosity (1300 RPM) as the emulsion was forming. Mixing was assisted with a spatula to scrape the sides of the main beaker. After 10 minutes of mixing, Phase D (Table 9) was added to the main beaker while mixing was continued. The emulsion was mixed for 5 minutes and the multi-speed stirrer stopped and extracted from the main beaker. The main beaker was weighed and deionized water was added to adjust the batch weight to 250 g. The multi-speed stirrer was positioned again on the main vessel and mixing continued until the emulsion was smooth and uniform. After the batch was finished, the emulsion was transferred into a 150 mL glass beaker to the level marked as 140 mL. This sample was left undisturbed overnight at 20° C. The control sample was prepared in a similar manner.

Physical Characteristics. The appearance and odor of sample and control were analyzed visually. Viscosity was measured on the sample kept undisturbed at 20° C. overnight. Spindle S 05 was used at 20 RPM on Brookfield Viscometer. Manual readings of viscosity and torque were taken after 1, 2, and 3 minutes. The spindle was removed after 3 minutes of reading. The same sample was used to measure pH using the pH meter. Five grams of the same sample were transferred on a disposable Petri dish (Fisher Brand, 60* 15 mm) and the inverted cover used to smear the sample to form a uniform film. The color of the film on the Petri dish was measured using Colorflex 45/0 Colorimeter.

Centrifuge Stability. The sample was transferred into two disposable conical tubes (Corning, 15 mL) to a total weight of 17 g. One tube was heated in a water-bath to 40° C. Both tubes were then centrifuged at 4000 RPM for 30 minutes. The control was not tested because there was a clear oil separation after mixing was stopped.

Accelerated Stability. The sample was transferred in two disposable conical tubes (Corning, 50 mL). One tube was marked and kept in the incubator set at 45° C. Readings on appearance, color, odor and stability of emulsion were taken on this sample at one week, 2 weeks, 3 weeks, one month, 2 months and 3 months data points. The other tube was kept in the freezer at −12° C. for 20 hours, and returned to the lab counter at RT for 4 hours. Readings were taken on appearance, color, odor and stability of emulsion. The sample was then kept in the incubator at 45° C. for 20 hours and returned to the lab counter for 4 hours. Readings were taken again on appearance, color, odor and stability of emulsion. This cycle was repeated for five times. The stability study for control was not initiated because there was a clear oil separation after mixing was stopped.

### Results

Physical Characteristics. The appearance of the sample emulsion was smooth, uniform and free of aeration. The odor was found to be fragrance-free and had a characteristic beany top note. The viscosity of undisturbed sample was found to be 8,440 cP at 20° C. with a torque of 84% after 3 minutes. The pH was found to be 4.69 at 20° C. The color of the sample was found to be L*78.66 a*0.46 b* 12.4. The control showed clearly that there was no emulsion formed without Lysofix. The control was transparent and had gel-like characteristics. The oil was not evenly dispersed in the control and had flocculated in areas. No further tests were conducted on the control.

Centrifuge Stability. None of the tubes that were centrifuged at 4000 RPM for 30 minutes showed any creaming, flocculation, settling or phase separation.

Accelerated Stability. The sample was kept at 45° C. and observed after 1 week, 2 weeks, 1 month, 2 months and 3 months. It did not show any change in appearance, color, or odor. No phase separation was observed at any time point. The sample kept for freeze/thaw cycle showed few oil droplets after the first cycle. The fine separated oil layer did not increase significantly over subsequent cycles. After 5 cycles, the emulsion showed no more change in appearance, color and odor, and there was no phase separation.

### Discussion

This study demonstrated that Lysofix can easily be used in a cold-processed emulsion and a satisfactory stability achieved with the careful selection of raw-materials and percentages of materials used. This process balances time and energy required to prepare emulsions. In this formulation, improvements can be made in optimizing the percentage of Lysofix used. In addition, change in particle size, and flocculation can be observed under a microscope for confirming stability. This study confirms that Lysofix is an effective natural emulsifier with the additional benefit of being able to be processed cold at room temperature, saving time and energy to the customer. It also opens opportunity to introduce heat-sensitive exotic materials for beauty and personal care market into a stable product.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

1. F. Zhang and A. Proctor. 1997. Rheology and Stability of Phospholipid-Stabilized Emulsions. JAOCS, Vol. 74, no. 7. SA-07-00397

We claim:

1. Stable emulsions, comprising:
(a) an oil phase;
(b) a water phase;
(c) an emulsifier consisting of a formulation of hydrolyzed natural non-GMO lecithin that is liquid at room temperature; and
(d) a preservative selected from the group consisting of natural extracts of species of the family Lamiaceae.

2. A stable emulsion as defined in claim 1, wherein the hydrolyzed lecithin consists of a 3% solution of lysophosphatidylcholines derived from soybean dissolved in glycerin derived from a vegetable source.

3. A stable emulsion as defined in claim 1, wherein the hydrolyzed lecithin consists of a clear liquid with a viscosity between 3,500 cP and 4,500 cP at 20° C. that flows slowly but easily by itself, and becomes more free-flowing when mixed with water.

4. A stable emulsion as defined in claim 1, wherein said emulsion does not separate upon storage at temperatures up to 45° C. for a minimum of 3 months.

5. A stable emulsion as defined in claim 1, wherein said species of the family Lamiaceae are selected from the group consisting of mint, rosemary, sage, savory, marjoram, oregano, hyssop, thyme, lavender, and perilla.
